(19) United States
(12) Patent Application Publication
(10) Pub. No.: US 2024/0206742 A1
(43) Pub. Date: Jun. 27, 2024

(54) In-Ear Health Sensor

(71) Applicant: Regents of the University of Minnesota, Minneapolis, MN (US)

(72) Inventors: Hubert Lim, St. Paul, MN (US); Anuj Bhardwaj, Austin, TX (US); Kashyap Patel, Hoboken, NJ (US); Claire Kaiser, Minneapolis, MN (US); Morgan Newhoff, Falcon Heights, MN (US)

(73) Assignee: Regents of the University of Minnesota, Minneapolis, MN (US)

(21) Appl. No.: 18/394,243

(22) Filed: Dec. 22, 2023

Related U.S. Application Data
(60) Provisional application No. 63/477,188, filed on Dec. 24, 2022.

(51) Int. Cl. A61B 5/02; A61B 5/00; A61B 5/026; A61B 5/107; A61B 5/145; A61B 5/1455; G06T 7/00

(52) U.S. Cl. CPC A61B 5/02007; A61B 5/0095; A61B 5/0261; A61B 5/1079; A61B 5/14546; A61B 5/14552; A61B 5/6817; A61B 5/6835; G06T 7/0016; A61B 2560/0468; A61B 2562/0204; A61B 2562/0238; A61B 2562/028; A61B 2562/046; G06T 2207/30104

(57) ABSTRACT

In some embodiments an apparatus for monitoring a physiological parameter is provided. The apparatus includes a housing configured to be placed into an ear canal of a subject. The apparatus further includes a sensor configured to measure at least one of blood vessel anatomy, a blood vessel dimension, blood flow properties, or a presence of a molecule in a blood vessel.

### RELATED APPLICATIONS

This application claims priority under 35 U.S.C. § 119 to U.S. Provisional Patent Application No. 63/477,188 filed on Dec. 24, 2022, titled “In-Ear Sensor with Energy Delivery and Integrated pMUT-ASIC Structures,” the entire contents of which is incorporated herein by reference.

### BACKGROUND

Detailed information about blood vessel activity in the ear drum can provide significant insight into the vital health state of individuals or people involved in activities requiring physical exertion, such as soldiers or warfighters. These blood vessels are uniquely positioned within the tympanic membrane (TM; also known as the ear drum) which is a thin tissue membrane that is a few hundred microns thick. However, current technologies used to characterize such blood vessels are limited due to the very small space of the ear canal region and the very small size of the blood vessels.

### SUMMARY

In some embodiments an apparatus for monitoring a physiological parameter is provided. The apparatus includes a housing configured to be placed into an ear canal of a subject. The apparatus further includes a sensor configured to measure at least one of blood vessel anatomy, a blood vessel dimension, or a presence of a molecule in a blood vessel.

In some embodiments an apparatus for monitoring a physiological parameter is provided. The apparatus includes: a housing configured to be placed into an ear canal of a subject; and an array including at least one of: a plurality of optical units, or a plurality of ultrasound transducers each including at least one transceiver channel, each of the ultrasound transducers including a piezoelectric micromachined ultrasonic transducer (pMUT) or a lead zirconate titanate (PZT)-based transducer, the array being coupled to the housing to provide imaging of a tissue of the subject.

In some embodiments a system for measuring a physiological parameter of a subject is provided. The system includes a plurality of pMUTs, each of which is configured to provide ultrasonic imaging of blood vessels within an ear of the subject. The system further includes a plurality of optical units. Each optical unit includes a light source configured to produce near-infrared light or mid-infrared light. The system further includes one or more electrodes that are configured to electrically stimulate a nerve of the subject. The system further includes a housing that is configured to be placed into an ear canal of the subject. The housing includes the plurality of pMUTs, the plurality of optical units, and the one or more electrodes. The system further includes a controller that is in communication with the plurality of pMUTs, the plurality of optical units, and the one or more electrodes.

In some embodiments a method for measuring a physiological parameter of a subject is presented. The method includes placing a health monitor inside an ear of the subject. The health monitor includes an array of a plurality of pMUTs and a plurality of optical units. Each optical unit includes a light-emitting diode and an optical sensor. The method further includes producing near-infrared or mid-infrared light using the LED of at least one of the plurality of optical units. The method further includes generating an image of blood vessels in the ear of the subject using at least one of the pMUT array or the optical unit. The method further includes analyzing the image to determine a physiologic parameter of the subject.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A provides a schematic overview of the anatomy of a tympanic membrane and a in vivo view of a tympanic membrane.

FIG. 1B provides a schematic overview of the anatomy of an inner ear.

FIG. 2 shows analytical and simulated assessment of ultrasound frequencies that can be achieved at varying diameters of transducers.

FIG. 3A shows an example piezoelectric micromachined ultrasonic transducer (pMUT) array module integrated with a complementary metal-oxide-semiconductor (CMOS) microcontroller.

FIG. 3B shows an example schematic side-view of the pMUT structure integrated with the silicon layers of the CMOS structure.

FIG. 3C shows an example of a single membrane pMUT.

FIG. 3D shows an example electrode arrangement of a pMUT circuitry with integrated optical units, including a square/rectangular pMUT membrane structure (left) and a circular pMUT membrane structure (right).

FIG. 4 shows an example system, including an in-ear health monitor in accordance with some aspects of the present disclosure.

FIG. 5A shows a first example arrangement of an in-ear health monitor apparatus.

FIG. 5B shows a second example arrangement of an in-ear health monitor apparatus.

FIG. 5C shows an example arrangement of an in-ear health monitor apparatus placed within an ear canal and adjacent to a tympanic membrane.

FIG. 6A provides a first example arrangement of an in-ear sensor including a pMUT component.

FIG. 6B provides a second example arrangement of an in-ear sensor including a pMUT component.

FIG. 7A shows an in-ear sensor, including a first example of an electrode arrangement.

FIG. 7B shows an in-ear sensor, including a second example of an electrode arrangement.

FIG. 7C shows an in-ear sensor, including a third example of an electrode arrangement.

FIG. 7D shows another example in in-ear sensor, including an example of an electrode arrangement.

FIG. 8A shows a flowchart outlining the steps of a first example method of using an in-ear health monitor.

FIG. 8B shows a flowchart outlining the steps of a second example method of using an in-ear health monitor.

FIG. 8C shows a flowchart outlining the steps of a third example method of using an in-ear health monitor.

FIG. 8D shows a flowchart outlining the steps of a fourth example method of using an in-ear health monitor.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

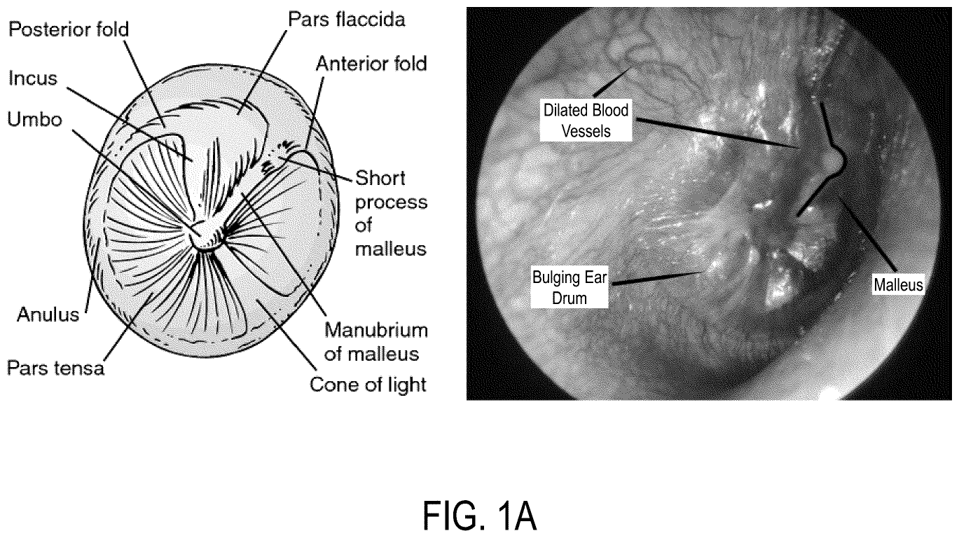

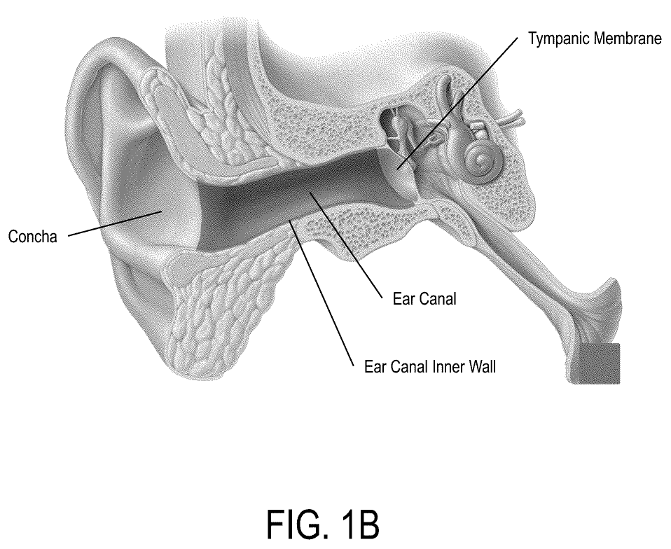

FIGS. 1A and 1B provide an overview of ear anatomy and a detailed example of an ear canal and tympanic membrane (TM; commonly referred to as the “ear drum”), characterized by a bulging ear drum/TM and dilated blood vessels. Detailed information about blood vessel activity in the ear drum can provide significant insight into the vital health state of individuals or people involved in activities requiring physical exertion, such as soldiers or warfighters. These blood vessels are uniquely positioned within the TM, which is a thin tissue membrane that is a few hundred microns thick, which enables direct visual access to the blood vessels (i.e., vessels are nearly on the surface), as demonstrated in the right panel of FIG. 1A. This situation provides a significant advantage for imaging the details of the blood vessels with approaches that typically require shallow depths and is not impaired or limited by skin or other tissue that can distort various imaging approaches. Furthermore, these blood vessels are highly sensitive to infections in the body or alterations in the body's physical or health state. For example, their dimensions and shapes can change (e.g., due to change in blood flow), they can turn redder (e.g., due to greater blood flow and vessel swelling), and additional vasculature/vessels can form throughout the membrane. These vessels are typically on the order of microns up to tens of microns in diameter, depending on their location and branching pattern on the TM. There are larger vessels that follow along the malleus middle ear bone area down to the umbo at the center of the TM with smaller branches going concentrically outwards to the outer rim of the ear canal, with also larger branches along the outer rim with more blood vessels within the ear canal as well. The blood vessels within the skin and tissue portion of the ear canal can also be imaged and can provide additional information, although these are less accessible and are not nearly on the surface as those in the TM.

Although the TM provides this unique and advantageous opportunity to accurately track a person's health or physical state, and even characterize changes in physiology and metabolism in the body (e.g., by tracking hemoglobin/blood oxygen, red/white blood cells, glucose, creatinine, calcium, potassium, lipids, vasoconstriction, impaired flow velocity, stopped flow, capillary perfusion, etc.), there have been limitations in technologies to accurately and continuously track these vessels due to the very small space of the ear canal region and the very small size of the blood vessels. Furthermore, the device must have high resolution, sensitivity, and precision to be able to recalibrate itself to continuously image the same vessels over time to be effective. Typical ultrasound, optical, and optoacoustic imaging methods have been insufficient to overcome this challenge with current approaches.

In this disclosure, novel technologies and techniques are provided which utilize combinations of approaches that have not yet been possible or have not been previously conceived and developed. Devices, systems, and methods are provided to track vasculature physiology and structure reliably and accurately over time to track a person's health or physical state. In some implementations, the technology involves an in-ear device or health monitor that is comfortably worn within the ear and which leverages sophisticated ultrasound imaging with high frequency and high resolution. In some implementations, implementations of the disclosed technology also include optical stimulation to achieve high resolution optoacoustic imaging. Furthermore, electrical stimulation of the ear region can be achieved using one or more electrode positioned within the ear canal and ear region (e.g., concha, ear lobe, outer ear regions). Such electrical stimulation can be used to modulate blood flow, adding a novel way to further enhance signals and create further differentiation between blood biomarkers. The electrical stimulation can be used to activate the vagal, trigeminal, or other nerves to alter blood flow that can alter blood flow and concentrations of blood biomarkers in different ways for different states. For example, vasculature can be evaluated and compared during passive blood flow states and modulated active blood flow states (e.g., varying blood flow via stimulation). In contrast, previous methods have focused on passive imaging of blood vessels. Altogether, this in-ear device can provide very high spatial and temporal resolution with higher sensitivity with strong and differentiating biosignals than has been possible previously.

Typical ultrasound technology can provide high frequency and high-resolution ultrasound but has not been manufacturable with the resolution necessary in a very small form factor (e.g., to fit within the ear region). In various embodiments, ultrasound transducers may include lead zirconate titanate (PZT)-based transducers, piezoelectric micromachined ultrasonic transducers (pMUTs), capacitive micromachined ultrasonic transducers (CMUTs), or other ultrasound transducers. In some embodiments, pMUTs may especially provide the capability to package adequate sensors as needed, and arrays of pMUTs may be mounted to a device which is sufficiently small so as to be placed close enough to the TM to obtain information from blood vessels and other structures in or near the TM.

In order to adequately beam form and image at high resolutions, it may be preferred to maintain channel spacing of pMUTs in an array at half or less of the wavelength of the ultrasonic frequency. The lateral resolution for ultrasound imaging improves with increased frequency and is typically equal to approximately 2.5 times the wavelength for most frequencies. In addition, the channels can be phased at specific delays to allow for high resolution beam forming, which at high frequency may require time-based resolution on the order of nanoseconds. This may require a much smaller channel spacing than bulk piezoelectrics are capable of attaining. However, pMUTs have significant advantages over existing bulk piezoelectric materials due to their low profile and high-resolution microstructure, enabled by precision manufacturing methods (e.g., stereolithography) currently employed in the semiconductor industry. Another advantageous attribute is the ability to directly bond or interconnect to an application specific integrated circuit (ASIC), allowing for high resolution array in two dimensions, providing volumetric ultrasonic imaging through direct control of each individual channel without the need for complicated interconnectors (e.g., ribbon cables, etc.). In order to achieve very high frequency and resolution, it may be advantageous to directly deposit the piezo-MEMS onto the ASIC, which may include a complementary metal-oxide-semiconductor (CMOS). Such design may allow for pixel density (or channel density) of less than 80 μm, which can enable 500 dpi (dots per inch) or greater, advantageous for high resolution and high frequency applications. Thus, the pMUT may be configured to transmit at a high frequency, such as 25-50 MHz, 50-75 MHZ, 75-100 MHz, 100-125 MHz, and so on.

The pMUTs can be manufactured using two primary formulations of film deposition, including aluminum nitride (AlN) and lead zirconate titanate (PZT). While AlN can be directly deposited onto CMOS due to its lower crystallization temperature using conventional methods, PZT typically cannot be due to its high crystallization temperature of 585° C. With standard methods, this crystallization temperature will cause reflow (and damage) of the metallization layers within the CMOS chip. However, with pulsed laser annealing or pulsed laser deposition methods, it is possible to localize heating upon the PZT film, causing crystallization where necessary, and avoiding damage to the CMOS substrate. PZT may be advantageous for in-ear (and other high resolution) detection applications due to its superior performance in pulse-echo ultrasound. Pulsed laser annealing can be accomplished using excimer lasers (or other laser technology) with masks (for area exposure) or galvanometers (local exposure) to heat specified regions (planar and depth), crystalizing the MEMS structures without heating additional areas beyond their metallization limits.

Table 1 provides non-limiting example parameters that can be achieved with ultra-high-frequency ultrasound (UHFUS), illustrating the potential for high-resolution blood vessel measurement.

TABLE 1

| | | |
|---|---|---|
| UHFUS Frequency | 70 MHz | 48 MHz |

| | | | | |
|---|---|---|---|---|
| Bandwidth | 29-71 | MHz | 20-46 | MHz |
| Axial Resolution | 30.0 | μm | 50.0 | μm |
| Lateral Resolution | 65.0 | μm | 110.0 | μm |
| Maximum Depth | 10.0 | mm | 23.5 | mm |
| Image Width (maximum) | 9.7 | mm | 15.4 | mm |
| Image Depth (maximum) | 10.0 | mm | 23.5 | mm |
| Focal Depth | 5.0 | mm | 9.0 | mm |

Table 2 provides non-limiting example parameters that can be achieved using UHFUS with varying excitation signals, illustrating the high axial resolution and signal to noise ratio (SNR) that can be achieved with UHFUS.

TABLE 2

| Excitation | Center | −6 dB Axial | | SNR | |
|---|---|---|---|---|---|
| Signal | Frequency | Resolution | | improvement | |
| Chirps | 70 MHz | 28.8 | μm | 16.4 | dB |
| Chirps | 46 MHz | 35 | μm | 15 | dB |
| Chirps | 40 MHz | 43 | μm | 12.5 | dB |
| Chirps | 31 MHz | 44 | μm | 14 | dB |
| Chirps | 17 MHz | 130 | μm | 11.9 | dB |
| 13-bit Barker | 40 MHz | 64 | μm | 11.14 | dB |
| 40 MPS | 40 MHz | 40 | μm | 16.02 | dB |
| 4-bit Golay | 30 MHz | 51 | μm | 7.26 | dB |
| 8-bit Golay | 30 MHz | 50 | μm | 11.47 | dB |
| 8-bit Golay | 30 MHz | 52 | μm | 13.32 | dB |

Tables 1 and 2 indicate the potential for high resolution blood vessel measurement using monolithically integrated PZT-based CMOS devices, in which frequencies of up to 100 MHz and higher are possible. This high resolution can enable microvessel imaging of vessels in the range of microns to tens of microns. Typically, using higher frequencies achieves shallower depth imaging capabilities. However, because the blood vessels in the TM are directly accessible at or near the surface, there is minimal attenuation and distortion. Thus, imaging the TM blood vessels or other surface vessels is well suited to these high frequencies.

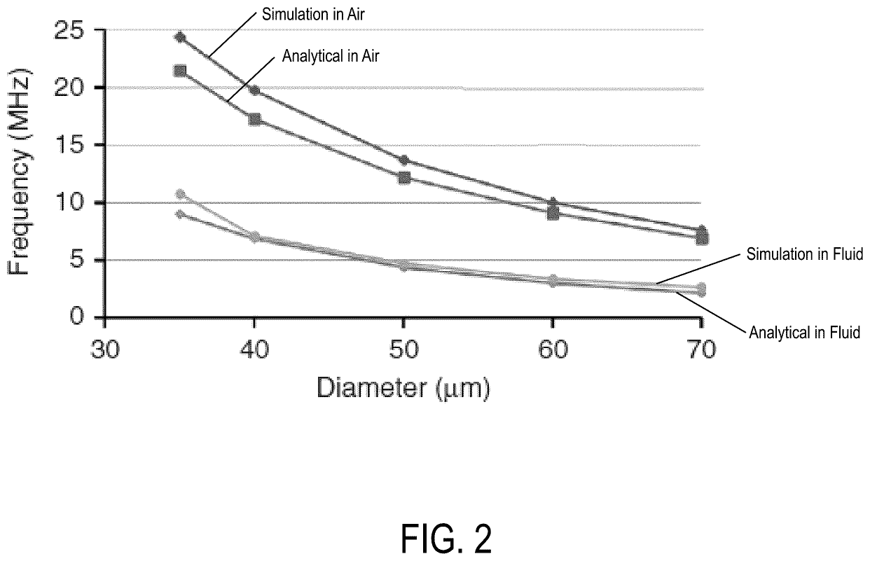

FIG. 2 shows an example of analytical and simulated assessment of the frequency achievable with varying diameters of piezo-MEMS membrane structures. For example, in some implementations, a membrane diameter smaller than 50 μm can be used to achieve frequencies adequate for high frequency ultrasound. One example of a monolithically integrated pMUT-CMOS product is an ultrasonic fingerprint sensor, which can be manufactured using AlN. AlN may be compatible with CMOS using conventional deposition and crystallization methods.

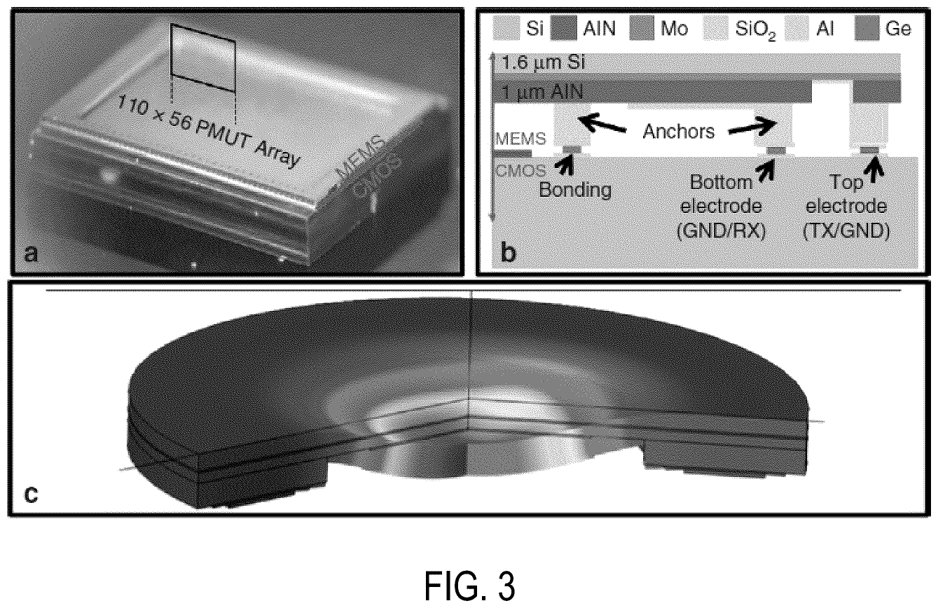

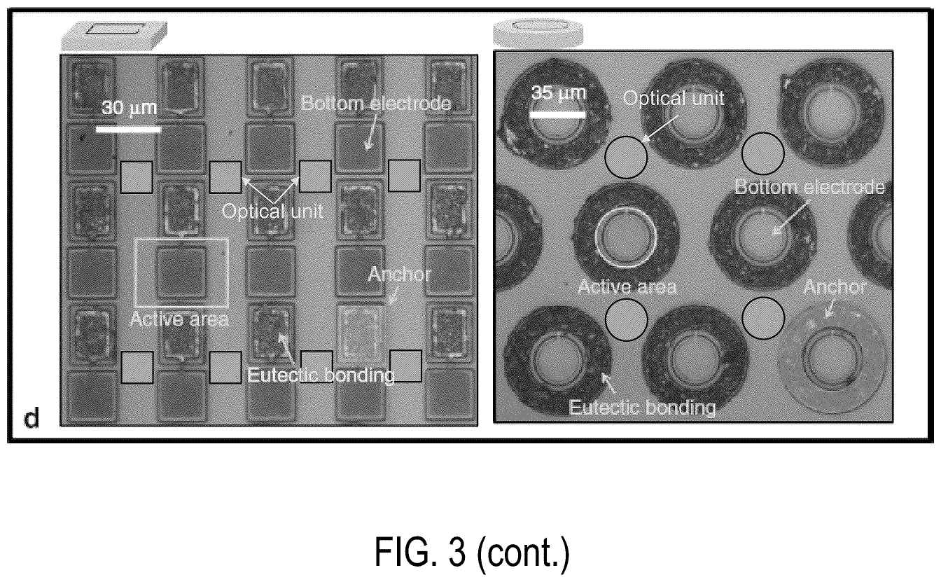

FIGS. 3A-3D illustrate the structure of example pMUT membranes and the channel spacing between them. In this example, this high-density arrangement of pMUTs has a pitch that is less than the 80 μm minimum pitch that can be obtained using known techniques such as interposers with wire bonding or through-silicon via methods. FIG. 3A shows an example piezoelectric micromachined ultrasonic transducer (pMUT) array module that is integrated with a complementary metal-oxide-semiconductor (CMOS) microcontroller. FIG. 3B shows a schematic side-view of an example pMUT structure integrated with the silicon layers of the CMOS structure. FIG. 3C shows an example single membrane pMUT, which operates in 3-1 mode with vertical displacement in the central section of the disk-like structure. FIG. 3D shows two example electrode layouts of the pMUT circuitry, including square/rectangular membrane structures (left) and circular membrane structures (right). As FIG. 3D shows, optical units may be integrated between ultrasound units in various patterns or in separate areas of the layout based on the desired application and resolution.

In some implementations, to achieve high sensitivity performance for in-ear detection upon a wearable platform, it may be advantageous to use high resolution monolithically integrated pMUT-CMOS devices, which may be made from PZT deposited film. Such devices may be enabled by advanced manufacturing methods such as pulsed laser annealing. In other implementations, the inner ear sensor may be designed using AlN, with or without scandium (Sn) doping, which can also achieve blood vessel imaging in the inner ear due to their relatively shallow depth below the skin. In addition, liquid coupling may be used. However, at very high frequencies and with high resolution ultrasound arrays, the need for liquid coupling may be reduced and in some implementations may not be necessary at all. Dry coupling methods may also be used to allow the ultrasonic sensor to couple with skin in the inner ear adequately enough to image blood vessels. For example, the in-ear sensor may directly contact the skin of the inner ear (e.g., tympanic membrane or ear canal wall).

In various embodiments, the pMUT capability can be integrated in a health monitoring sensor with a very small form factor. For example, the sensor may be limited to 20 mm, 15 mm, 10 mm, 9 mm, 8 mm, 7 mm, 6 mm, 5 mm, 3 mm, 2 mm, 1 mm, or smaller in one or more dimension, including for example the dimensions of the portion of the sensor device which interfaces with the TM. This small form-factor can allow the health monitoring sensor to be placed into the ear as part of an in-ear health monitoring device that can fit snugly into the ear canal. In some implementations, the pMUT capability can be combined with advanced ear imaging and 3D printing techniques to build in-ear fitted devices that are customized for each subject in a scalable way.

In some configurations, the device may be implemented using an ultrasound imaging modality, enabling imaging of the blood vessels and surrounding structures. Such imaging may include monitoring changes in vessel dilation and constriction and blood flow dynamics. In some configurations, to further enhance the imaging capabilities, the in-ear device also includes an optical light source (e.g., light-emitting diodes (LEDs)). The light source can be used to implement the low-cost and scalable ability to provide near-infrared (NIR) or mid-infrared (MIR) light to the ear drum. This light source can be used optical imaging or optoacoustic imaging, which may include spectroscopy. The light source or ultrasound source may also be used for perturbation of the tissue in order to track the vibrations through the tissue (e.g., similar to elastography methods). LEDs can be turned on and off very quickly, for example on a scale of tens of nanosecond, which is short enough to enable optoacoustic imaging with spatial resolution in the microns range. This optical excitation can be combined with the very high transmit/receive ultrasound imaging frequencies (e.g., 25-50 MH, 50-75 MHZ, 75-100 MHz, or 100-125 MHz) to achieve sufficient spatial resolution for TM vasculature imaging. The optoacoustic imaging using a light source may also include laser-based methods.

One drawback with LEDs, as compared to laser-based methods, for example, is that LEDs have much weaker energy in a focused location (e.g., LEDs lack coherence and are not monochromatic sources). However, by acquiring data in a fast and repetitive way, many averages can be performed (e.g., 10, 100, 500, or 1000 times) within a short time period. For example, a 5 kHz repetition rate can enable an image or frame rate of 10 Hz, which is fast enough to fit within a breathing cycle. Using multiple averages, the SNR can be greatly enhanced even for low optical energies. Increasing the SNR of the light source in the tissue can allow for thermal expansion and a vibration/shock waves through the tissue. For example, short pulses (e.g., on the order of tens of nanoseconds) may be generated using the light source, and the resultant ultrasonic waves generated can be measured by the ultrasound imaging sensors. Advantageously, this approach can be used to sense molecules in the blood stream, such as glucose. For example, glucose has good absorption properties in the MIR range (approximately 3 μm-30 μm wavelength range).

Current technologies typically use NIR because there are LED technologies available in that range, but they are limited since stronger more distinct absorption signals occur for MIR. However, MIR is limited in depth of transmission in tissue and greatly affected by skin and varying tissue surfaces. Furthermore, LEDs for MIR are just recently being developed that can span the MIR range of roughly 3 microns to 10 microns wavelengths (as high as 30 microns), where good glucose monitoring can occur around 10 microns (similar to the wavelength range for other molecules that require MIR for better resolution), whereas the NIR range of less than 3 microns down to a few hundreds of nanometers is not as well suited for such measurements. Fortunately for the TM, using wavelengths in the MIR range is possible because there is very little optical attenuation loss to excite the blood vessels, requiring less energy to be used. When combined with the high resolution and sensitivity of the described pMUT ultrasound sensing procedures disclosed herein, it is possible to perform imaging of molecules within the blood vessels with high resolution and sensitivity.

Simple B-scanning can be performed and Fourier domain spectroscopy, as well as more complex imaging reconstruction methods, may be employed. Use of LEDs also enables safe levels that are not as dangerous as typical class IV laser devices. For measurements of hemoglobin in erythrocytes and measurements in other blood cell types, NIR is still effective and thus both NIR and MIR optical ranges of stimulation may be used to provide more comprehensive characterization of the blood flow pattern and/or of molecules flowing through the blood. Thus, in some embodiments, NIR can be leveraged to especially image certain molecules that are still detected or imaged by NIR better than MIR. MRI may also be included to image other molecules of interest.

An advantage of certain embodiments of the present procedures is that using the disclosed advanced ultrasound imaging can more specifically focus and target individual blood vessels rather than imaging an averaged and diluted intensity across bulk tissue, which may encompass multiple vessels. Thus, it is possible to detect and sense finer changes in blood molecule concentrations (e.g., through blood vessels or within surrounding tissue, like the interstitial space) to differentially detect changes in the physiological or physical state of an individual. This functional information can be obtained in addition to information such as heart rate, oxygenation levels, blood flow, etc. that may be obtained from the structural imaging information.

As a non-limiting example, optical pulses can be in the NIR and MIR ranges (e.g., 700 hundred nanometers to 15 micrometers wavelength), with pulse durations of 5-300 nanoseconds and energies of 10-500 microjoules. Each LED can have a width and length of about 100 microns (e.g., in a range of 75-125 microns), though smaller and/or larger LEDs may be used, such as 10 microns, 25 microns, 50 microns, and so on or up to 1 mm, based on the application.

It is possible to cause hearing by using optoacoustic stimulation of the ear drum or ultrasound stimulation around the head. Therefore, in some implementations, it may be advantageous to use much weaker energy stimulation signals for imaging purposes so that the subject does not experience sound sensations as a result of the sensor activity. For this reason, the described approach may use LED signals with less energy and extensive averaging, as compared to existing methods. As a non-limiting example, the LEDs may use energies of <5 microjoules or 5-10 microjoules per pulse. The LEDs may be activated for pulse lengths of 10 ns with a 532 nm laser. In other embodiments the pulse duration may be set as 0.5-5 ns, 5-10 ns, 10-20 ns, 20-30 ns, 30-40 ns, 40-50 ns, 50-100 ns. The LED wavelength may be set between 450-500 nm, 500-550 nm, 550-600 nm, or 600-650 nm. The approach may also target high-resolution imaging of specific/particular blood vessels to advantageously avoid sound sensations. Similarly, using much higher frequency ultrasound pulses and imaging can prevent sounds from being heard from the ultrasound that vibrates head fluids/tissue which might otherwise lead to sound perception. For example, the ultrasound may use frequencies of 5-10 MHz or >10 MHz and may include ramping or smoothing of sharp transitions.

Additionally, in some implementations, the in-ear sensor system can leverage neuromodulation of the vagus and trigeminal nerves or receptors around the ear regions to modulate blood flow and heart rate. While it is known that electrical stimulation of these nerves can alter blood flow dynamics, the present device provides a combination of electrical stimulation of nerves to perturb the blood vessels while imaging such vessels, which has not been possible with existing technology. This provides even richer information of blood flow dynamics and molecular transmission than is possible in a passive state of imaging. Not only can the described systems and methods measure greater variations in vessel dilation and constriction at different time points, which will be different for ill or healthy states or in different physical states, but also the flow of different molecules, which will vary due to their mass/viscosity patterns through blood for different blood flow rates. The latter affects their density in the blood and thus their absorption patterns. This information can then be used in various ways to further refine concentration measurements in the blood that are sensitive to different molecules, depending on the blood flow rate.

Electrodes can be positioned in different locations around the ear and can be stimulated with electrical current to activate a nerve or receptor in the ear region. For example, electrodes can be used to excite the vagus nerve, the trigeminal nerve, the facial nerve, the somatosensory nerve, or combinations thereof to alter the blood flow state. This concept can be further viewed in systems engineering of having an input and output signal but changing the transfer function of the system in different ways, and thus various transfer functions can be identified, corresponding to different molecules based on the varying input and output data.

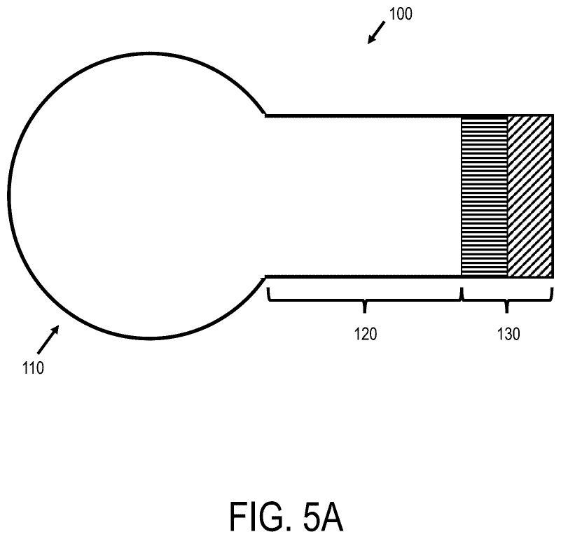

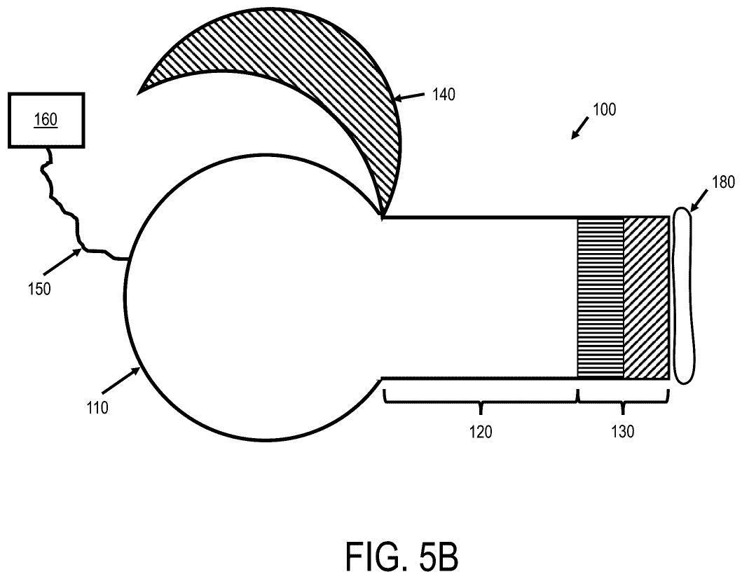

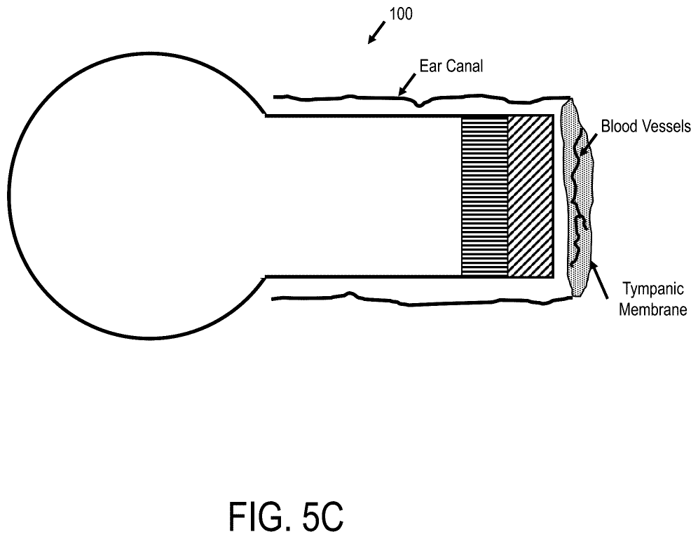

In a non-limiting example, each electrode may be about 1 mm in diameter (for circular electrodes) or 1 mm in length and width (for rectangular electrodes). In some configurations, smaller electrodes (e.g., 0.5 mm) can be used. In some implementations, the electrodes may have diameters or lengths/widths of up to 10 mm for greater surface area to drive more current with greater comfort. Current levels can span a range of 100 microamperes up to 5 mA with pulse durations from 20 microseconds to 2 milliseconds. Different biphasic or monophasic pulses can be used in monopolar, bipolar, or tripolar configurations to direct current flow in customized ways for greater activation and comfort. Various numbers (e.g., up to 10, or even more) of electrodes can be positioned in different locations in the ear canal, concha, and outer ear regions. A ground electrode may also be positioned within the ear canal, concha, or outer ear regions or around the back side of the ear. In some implementations, a hook (e.g., similar to a hearing aid, see FIG. 5B) can wrap around the ear to secure the device into or onto the ear. In other configurations, the device may be fully positioned within the ear canal (e.g., similar to an ear plug, see FIG. 5C).

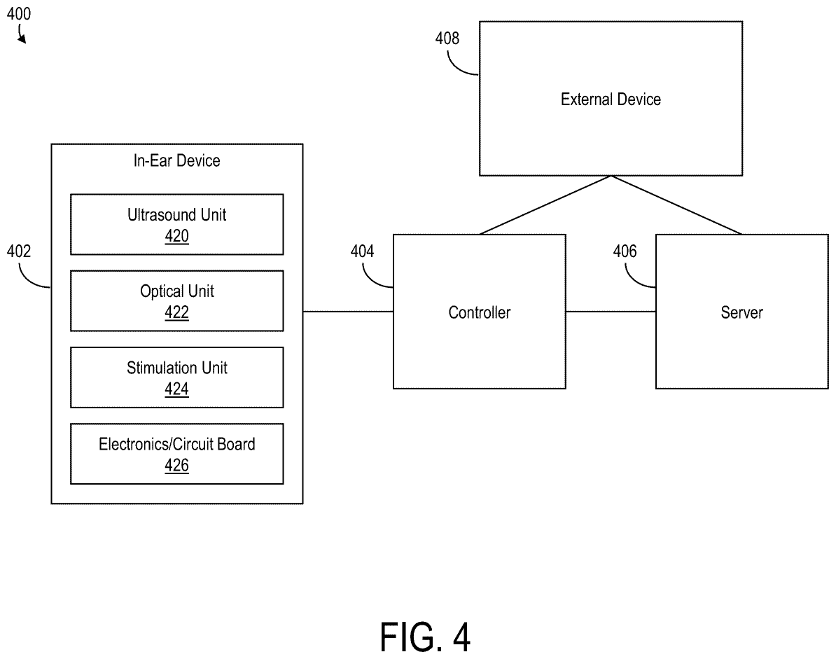

FIG. 4 illustrates an example monitoring system 400, which may be used in accordance with the present disclosure. The system 400 may include an in-ear device 402 that houses an ultrasonic unit or ultrasound unit 420. The ultrasound unit 420 may include one or more ultrasound or ultrasonic transducers, which may be configured as ultrasound transmit transducers, ultrasound receive transducers, ultrasound transmit/receive (or “transceive”) transducer. For example, the ultrasound unit 420 may include some combination of such transducers arranged in an array, which may have high 2D resolution. As previously described, the ultrasound transducers may include one or more piezo-MEMS, such as pMUTs. The ultrasound transducers may also include PZT materials, such as a PZT-based CMOS device or other PZT ultrasound transducer. The ultrasound transducers may also include CMUTs or other ultrasound transducers. In some embodiments, an array of ultrasound units may include some combination of pMUTS, PZT-based transducers, and CMUTs.

The in-ear device 402 may further include one or more optical units 422. The optical units 422 may include a light source, such as LEDs or lasers, to provide optical stimulation, optical imaging, or optoacoustic imaging. For example, the optical stimulation may provide optoacoustic imaging along with the ultrasound units 420 that can detect ultrasound or sound generated in response to the optical stimulation. The optical units 422 may further include optical detectors or sensors, such as photodiodes, photoconductive sensors, or photovoltaic cells. In some configurations, the optical units 422 can provide optical stimulation and optical imaging of the tissue.

The in-ear device 402 may also include a stimulation unit 424 to provide nerve stimulation. In some embodiments, the stimulation unit 424 may include one or more electrodes to provide electrical stimulation. The stimulation unit 424 may include an optical light source (e.g., visible light, NIR, MIR, etc.) to provide optical stimulation. The stimulation unit 424 may include a magnetic field generator or magnetic stimulator to provide magnetic stimulation. The stimulation unit 424 may also include an actuator, such as a piezoelectric actuator, eccentric rotating matt actuator, linear resonant actuator, or other haptic device. The actuator may be configured to produce vibrations. The stimulation unit 424 may also be configured to produce sound stimulation, using a speaker or other electroacoustic transducer. The stimulation unit 424 may include an ultrasound transducer configured to produce ultrasound stimulation. In some embodiments, vibrational or ultrasound stimulation may also be provided by the ultrasound unit 420.

The in-ear device may additionally include electronics 426 or circuit boards, such as printed circuit boards (PCB), for controlling or measuring feedback from the ultrasound units 420, optical units 422, stimulation units 424, or other electrical components.

As illustrated in FIG. 4, the in-ear device 402 can be controlled via Bluetooth, WiFi, or other wireless or wired connection that connects the device to a controller 404 or base device. The controller 404 can store data, perform large scale data analyses, or transfer the data to another server 406 or processor for storage or analysis. In some examples, a wired external device 408 may be used (e.g., hung around the neck, worn as headphones or helmet, or otherwise placed on or near the body) if greater power requirements are needed. Such external device 408 may provide an additional power source. The controller 404 or external device 408 may be connected to a server 406 (e.g., cloud, internet, large scale database, processor) via a wired or wireless (e.g., Bluetooth, WiFi). Data can be stored or processed on the server 406. For example, the data may be processed using artificial intelligence or deep learning networks in pseudo real-time using an offline- or internet-based algorithm.

Various methods of tracking of blood vessels with imaging may be used. The imaging may be achieved using ultrasound, optical sensors, or a combination thereof. For example, imaging measurement parameters may include dimensions, pulsations, shapes, branching patterns, newly formed or altered vasculature, blood flow (e.g., velocity and volume), number of vessels, tension or elasticity of blood vessel walls using elastography methods (e.g., to assess heart health correlated with elasticity of blood vessels and blood pressure measurements), heart rate measurements, etc.

Various methods of tracking of blood vessels with optical or optoacoustic imaging may also be used. For example, the optical or optoacoustic imaging may be used to measure absorption of various molecules in the blood, oxygenation levels, formation or alteration of blood vasculature, swelling of blood vessels and surrounding tissue (e.g., to track the initiation of an infection/ill state before body symptoms even form), prediction of stress, fear, or anxiety states due to vasculature and blood molecular changes (e.g., before behavioral changes occur in individuals or warfighters to help them cope with them sooner or maintain better focus sooner), assessing physical limits that are possible during training (e.g., to push the limits in warfighters or during extreme exercising), tracking diet and healthy eating states, tracking various disease states (e.g., diabetes, drug/alcohol abuse states, inflammatory conditions, metabolic or digestive issues, etc.), or other conditions.

### In-Ear Health Sensor Apparatus

FIGS. 5A-5C show various embodiments of example in-ear health sensors. In certain embodiments, an in-ear health sensor apparatus 100, which incorporates the above-described technology, may include a body portion 110, a stem portion 120, and a terminal portion 130 (FIG. 5A). The body 110 and/or stem 120 portions may include components such as a power supply (e.g. battery), electronics (e.g. a processor and/or memory), and/or communications (e.g. wired or wireless, such as Bluetooth or WiFi) components.

The terminal portion 130 may include source and/or sensor components along with other electronics situated within an external housing (which may be part of the stem portion 120). For example, the terminal portion 130 may include: an ultrasound transducer layer (e.g., a piezo-MEMS (pMUT) layer); one or more ASICs, such as an ultrasonic pulse beam forming and receiver pass-through ASIC or an analog front end (e.g. for amplification and/or digitization); one or more optical units, which may include LEDs, photodiodes, optical detectors, or a combination thereof; and a substrate or printed circuit board (PCB) layer.

In some embodiments, as shown in FIG. 5B, the apparatus 100 may include a behind-the-ear hook 140 to help secure the device to the subject's ear. In certain embodiments, the hook 140 may house additional components such as power supply, electronics, or communications components. In various embodiments, the apparatus 100 may fit into the concha of the ear or may wedge behind the tragus to help secure the apparatus 100 into place, with or without the hook 140 for additional support. In particular embodiments, the apparatus 100 may be configured (e.g. with touch-control surfaces) such that touching the device or a portion of the ear (e.g. the tragus region) activates, deactivates, or adjusts the apparatus 100. In some embodiments, the apparatus may be in communication 150 (in a wired or wireless manner) with an external electronics unit 160 or external device, which also may house additional components, such as power supply, electronics, or communications components. The external electronics unit 160 may be worn on the subject's body (e.g. clipped onto the body, clothing, or helmet, or on a chain or lanyard around the neck) or may be in a nearby location (e.g. a vehicle or building); locating the external electronics unit 160 in a location that is not coupled to the subject's body may be particularly advantageous if the communication 150 is wireless. In some embodiments the terminal portion 130 may be coupled to the tympanic membrane via a coupling medium 180 such as a gel to provide greater sensitivity. In certain embodiments, imaging of blood vessels in the ear canal inner wall is also possible although the resolution of such images may be lower than that of the tympanic membrane to image vessels at greater tissue depth.

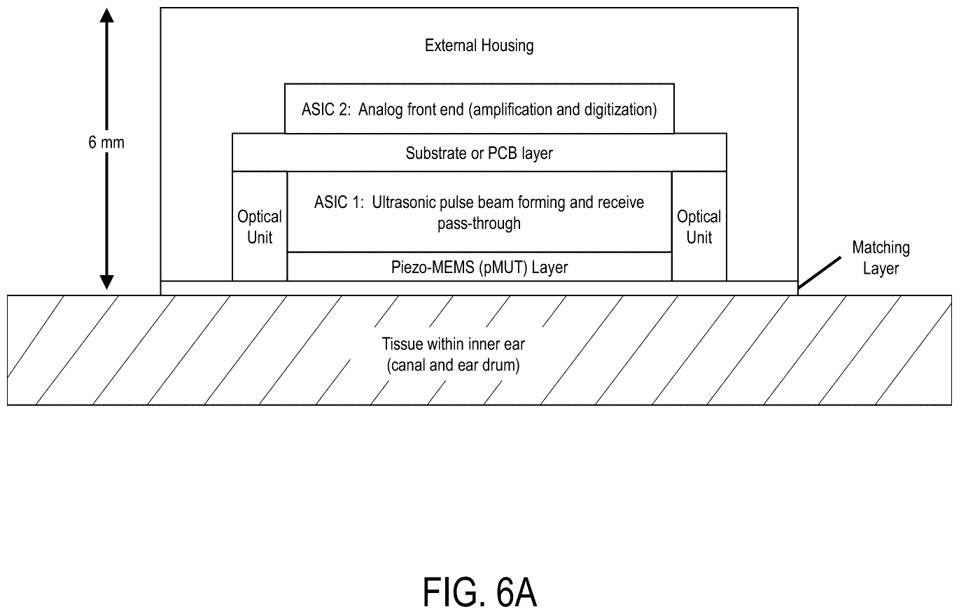

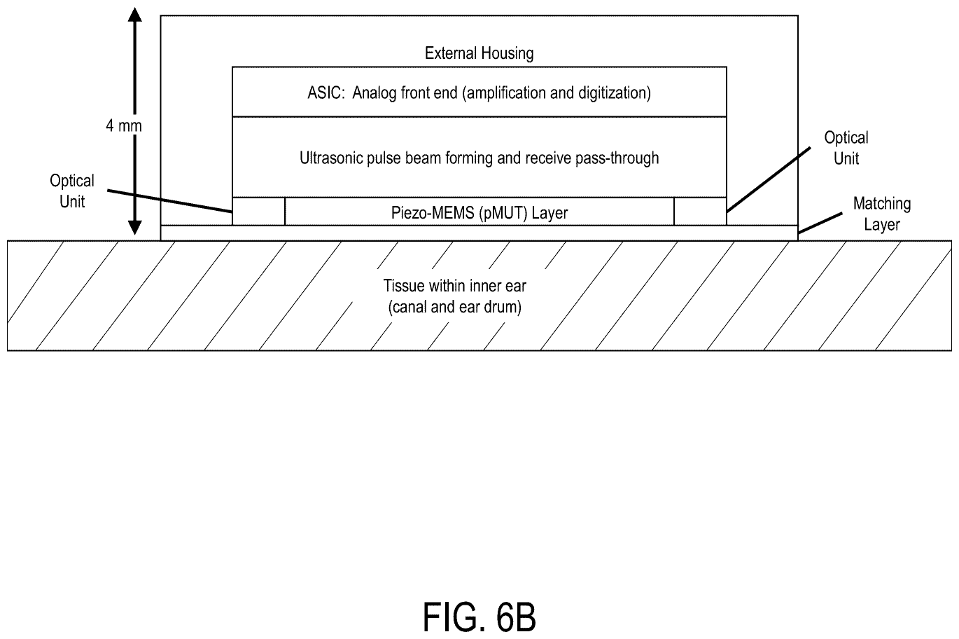

FIGS. 6A and 6B show schematic representations of the electronic elements that may be included in the apparatus 100 in two example arrangements. In the first example, shown in FIG. 6A, two ASIC layers are combined using a substrate of printed circuit board (PCB) or an interposer. The first ASIC includes an ultrasonic pulse beam forming and receive pass-through. Piezo-MEMs can be directly deposited onto this first ASIC layer. The second ASIC can include an analog front end for amplification and digitization. In the second example, shown in FIG. 6B, a single, fully integrated ASIC is used. This integrated ASIC includes an analog front end for amplification and digitization and an ultrasonic pulse beam forming and receive pass-through. In some examples, the integrated ASIC does not require an interposer or flip-chip to make contact between the ASIC and pMUT. This allows for a smaller footprint, greater robustness, and high throughput direct-bonding manufacturing techniques. Direct bonding can be achieved through pulse-laser annealing techniques to avoid damage through re-crystallization of PZT material.

The piezo-MEMS layer may be configured to be placed near or in contact with the tissue of interest (e.g., the ear canal or tympanic membrane). An optional matching or coupling layer (such as a gel) may be placed between the pMUT layer and the tissue. A light source, such as one or more LED lights, may be placed adjacent to the pMUT layer such that the light source is placed near or in contact with the tissue in use. In some embodiments the LEDs may be interspersed between the pMUTs and in other embodiments the LEDs may be placed adjacent to the pMUT array.

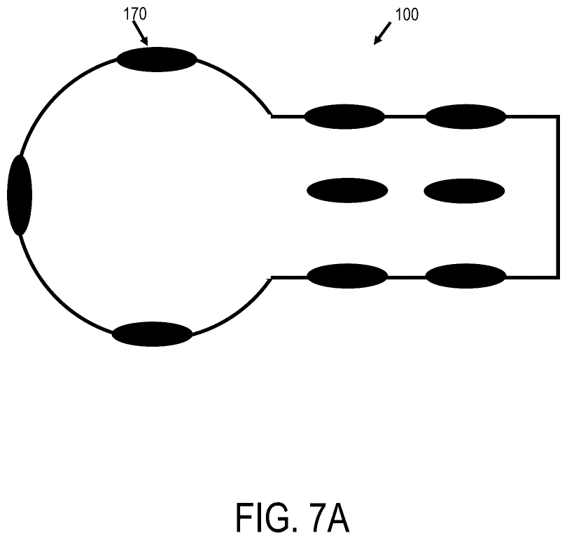

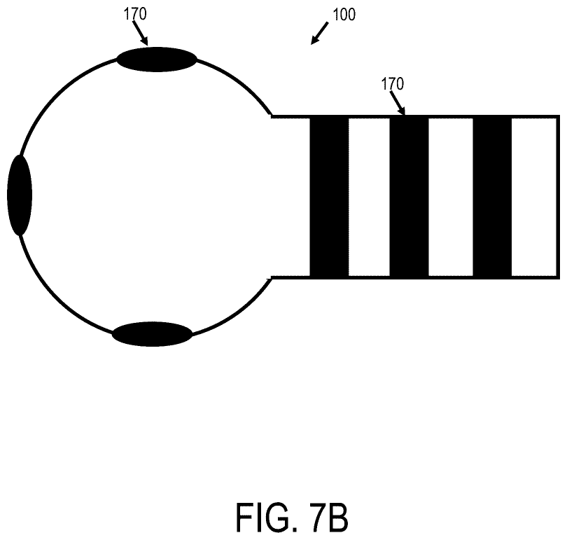

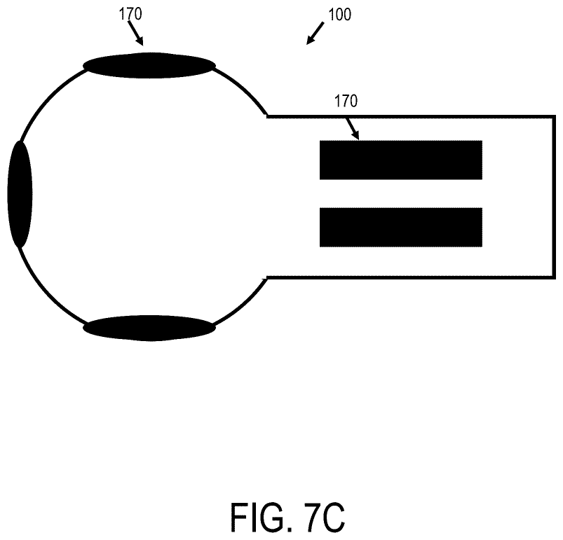

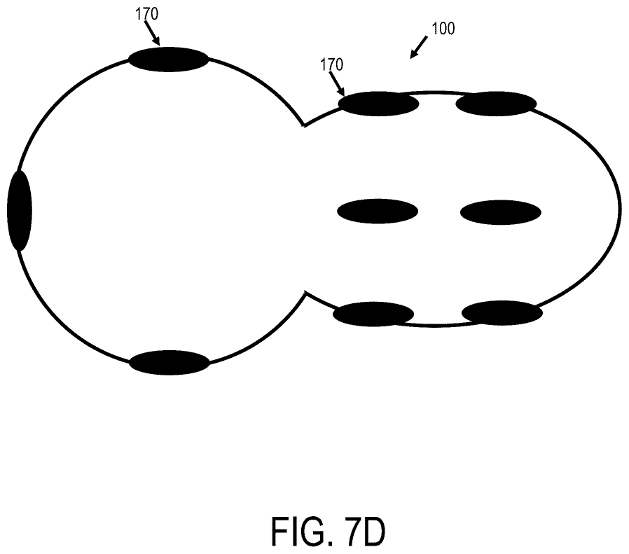

In various embodiments, the apparatus 100 may include one or more electrodes 170 on the outer surface. Non-limiting examples of various electrode arrangements are provided in FIGS. 7A-7D. In use, the electrodes 170 can stimulate the vagus, trigeminal, other sensory or touch nerves, or a combination thereof. The electrodes 170 may be formed as discrete spots (as shown in FIGS. 7A and 7D, for example). The electrodes 170 may also be formed as rings that encircle parts of the apparatus. For example, FIG. 7B shows electrodes 170 formed as rings encircling the stem portion 120 of the apparatus 100 to provide improved surface area for greater contact with the surrounding tissue. In some embodiments the electrodes 170 may be elongated in a longitudinal configuration, as shown in FIG. 7C. Other electrode configurations can also be used based on design requirements and the target application.

In some embodiments, the apparatus 100 may have a more curved or rounded shape, as shown in FIG. 7D for example. The apparatus 100 may also have varying lengths (e.g. shorter or longer), to better conform to various subjects' ears and ear canals. In other embodiments, the body of the apparatus 100 may be customized for a particular subject, e.g. by performing a 3D scan or generating a mold from within the subject's ear, or a variety of different sized body portions may be made to fit the majority of ear sizes and shapes.

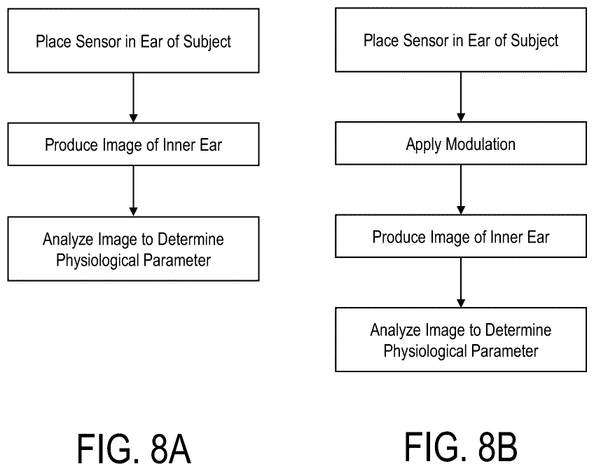

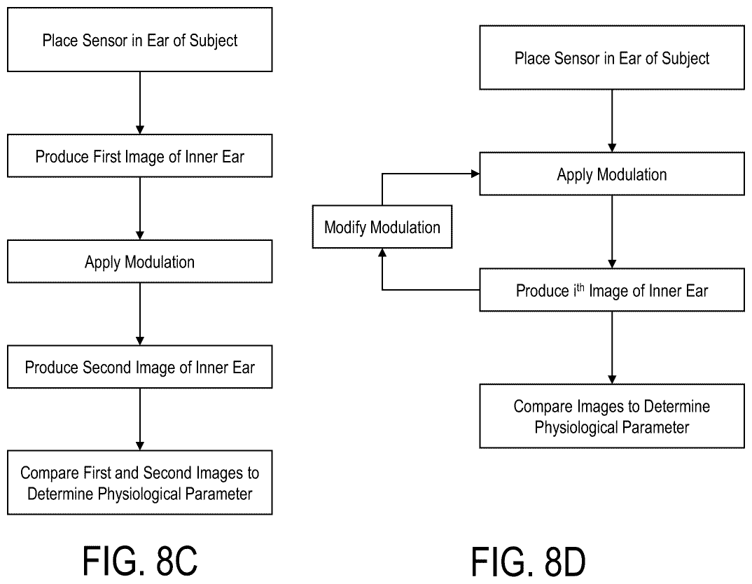

FIGS. 8A-8D show flowcharts that illustrate example methods of using the apparatus and system previously described. As shown in FIG. 8A, for example, the health sensor can be placed into an ear of a subject to produce images of the inner ear (e.g., tympanic membrane, ear canal). Images can be produced using the ultrasound unit, in which ultrasound waves are transmitted and received by the ultrasound unit to produce ultrasonic images. Images can additionally or alternatively be produced using the optical unit by transmitting and receiving optical signals (e.g., light source) to produce optical images. The images can be analyzed in order to determine a physiological parameter. For example, the images can be used to analyze the structure or size of the blood vessels. Imaging can be repeated as desired to track changes over time (e.g., throughout the course of an exercise regimen, over the course of treatment, etc.) or to provide image averaging. Thus, analysis may include comparison of images at various timepoints or analysis of images averaged over time.

In other configurations, as shown in FIG. 8B, physiological modulation can be applied in conjunction with (e.g., prior to or during) image acquisition. As a non-limiting example, modulation may include optical excitation (e.g., NIR light, MIR light) using a light source (e.g., LEDs) of the optical unit. Modulation may also include nerve stimulation (e.g., vagus nerve, trigeminal nerve, or sensory nerve) using electrical stimulation produced by an electrode. Modulation may also include ultrasound stimulation of the tissue or nearby nerves (e.g., producing vibrations in the tissue or exciting the vagus nerve) produced by the ultrasound unit. Modulation may also include a simultaneous or consecutive combination of optical, electrical, or ultrasound stimulation. In some configurations, modulation and image acquisition can be repeated to obtain many images to increase image averaging for analysis of images with improved SNR. As previously described, image acquisition may include use of an ultrasound unit, an optical unit, or a combination thereof. Thus, ultrasonic images, optical images, optoacoustic images, or a combination thereof may be produced.

In some configurations, the physiological parameter may be determined based on changes in the images over time or varying modulation parameters. For example, as shown in FIG. 8C, a first set of images can be produced without modulation, and a second set of images can be produced with modulation (e.g., optical, electrical, or ultrasound). The images can be compared to identify changes (e.g., vessel dilation, blood flow, molecular concentration, and so on) that are precipitated by the modulation. In other embodiments, parameters of the modulation can be changed (e.g., light wavelength, pulse duration, current level, spatial location, and so on), as indicated in FIG. 8D.

FIGS. 8A-8D provide several examples of methods that may be used to determine one or more physiological parameters of a subject. However, other methods may also be used based on the desired application. The system, which may include various combinations of ultrasonic imaging, optical imaging, and optoacoustic imaging paired with or without various combinations of optical stimulation, ultrasound stimulation, and electrical stimulation, provides flexibility to alter the transfer function of the physiological system to interrogate many different physiological parameters. These parameters may include blood flow changes (e.g., velocity, volume), number of vessels, dimensions and shapes of vessels, swelling of vessels or surrounding tissue, newly formed or altered of vasculature, presence of infection, changes in metabolism, hemoglobin levels, blood oxygenation, concentration or red or white blood cells, concentration of other molecules (e.g., glucose, creatinine, calcium, potassium, lipids), vasoconstriction, impaired flow velocity, stopped blood flow, capillary perfusion, changes in blood flow dynamics, pulsations, vessel branching patterns, tension or elasticity of blood vessel walls (e.g., using elastography methods), heart rate, prediction of stress, assessment of physical limits, tracking diet, assessing disease states (e.g., diabetes), and so on.

For example, in some embodiments, ultrasound images may be produced in order to characterize the anatomy of blood vessels in an ear region of a subject. Ultrasound images may also be used to measure blood flow patterns and rates. In some embodiments, optical images may be produced to characterize blood vessel anatomy and blood flow patterns. Optical images may also be used to measure a presence or concentration of biomarkers or molecules in the blood within a blood vessel. As non-limiting examples, such molecules may include oxygen, glucose, creatinine, cytokines, white blood cells, etc. In some embodiments optoacoustic images may be produced. Such optoacoustic images may rely on ultrasound or sound detection to measure or characterize vibrations caused by thermal expansion of the tissue precipitated by optical stimulation (e.g., LED or laser). These images can provide characterization of vessel anatomy, blood flow, and blood contents (e.g., molecule presence or concentration) with a high spatial resolution. In some embodiments, ultrasound, optical, or optoacoustic imaging can be combined with electrical stimulation that modulates the physiological system. For example, electrical stimulation of a nerve can modulate heart rate or blood flow patterns, which may also change biomarker concentrations in the blood within various vessels.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

What is claimed is:

1. An apparatus for monitoring a physiological parameter, the apparatus comprising:
a housing configured to be placed into an ear canal of a subject; and
a sensor configured to measure at least one of blood vessel anatomy, a blood vessel dimension, blood flow properties, or a presence of a molecule in a blood vessel.

2. The apparatus of claim 1, wherein the sensor comprises an array of at least one of a plurality of ultrasound units or a plurality of optical units.

3. The apparatus of claim 2, wherein the sensor is configured to produce at least one of an ultrasound image, an optical image, or an optoacoustic image.

4. The apparatus of claim 2, wherein each of the plurality of ultrasound units comprises an ultrasound transducer, the ultrasound transducer comprising at least one of a PZT-based ultrasound transducer, a piezoelectric micromachined ultrasonic transducer (pMUT), or a capacitive micromachined ultrasonic transducer (CMUT).

5. The apparatus of claim 2, wherein each of the plurality of optical units comprises a light-emitting diode (LED).

6. The apparatus of claim 5, wherein each of the plurality of optical units further comprises an optical detector or photodiode.

7. The apparatus of claim 2, further comprising one or more electrodes configured to electrically stimulate a nerve or receptor in an ear region of the subject.

8. The apparatus of claim 7, wherein the nerve is at least one of a vagus nerve or a trigeminal nerve of the subject.

9. The apparatus of claim 2, further comprising a stimulation unit configured to stimulate a nerve or receptor in an ear region of the subject in order to modulate at least one of a heart rate or blood flow rate in the subject.

10. The apparatus of claim 9, wherein the stimulation unit is configured to produce at least one of magnetic stimulation, electrical stimulation, vibration stimulation, or ultrasound stimulation.

11. An apparatus for monitoring a physiological parameter, the apparatus comprising:
a housing configured to be placed into an ear canal of a subject; and
an array comprising at least one of:
a plurality of optical units, or
a plurality of ultrasound transducers each comprising at least one transceiver channel, each of the ultrasound transducers comprising a piezoelectric micromachined ultrasonic transducer (pMUT) or a lead zirconate titanate (PZT)-based transducer,
the array being coupled to the housing to provide imaging of a tissue of the subject.

12. The apparatus of claim 11, wherein the array comprises the plurality of optical units and the plurality of ultrasound transducers, each optical unit comprising a light source configured to produce at least one of near-infrared light or mid-infrared light to provide optoacoustic imaging.

13. The apparatus of claim 11, wherein each optical unit comprises a light source configured to produce at least one of near-infrared light or mid-infrared light and an optical detector to provide optical imaging.

14. The apparatus of claim 11, wherein the array comprises the plurality of ultrasound transducers comprising a plurality of pMUTS to provide ultrasonic imaging.

15. The apparatus of claim 14, wherein each of the plurality of pMUTs is configured to transmit frequencies between 50-75 MHz.

16. The apparatus of claim 14, wherein each of the plurality of pMUTs comprises at least one of aluminum nitride (AlN) or PZT deposited onto a complementary metal-oxide-semiconductor (CMOS) substrate.

17. The apparatus of claim 11, further comprising one or more electrodes configured to electrically stimulate a nerve or receptor in an ear region of the subject.

18. The apparatus of claim 11, wherein the plurality of ultrasonic transducers comprises a monolithically integrated pMUT-CMOS device.

19. The apparatus of claim 11, wherein the array of ultrasound transducers has pitch of less than 80 μm.

20. The apparatus of claim 11, wherein the tissue comprises at least one blood vessel in an ear of the subject.

21. The apparatus of claim 20, wherein the at least one blood vessel is disposed within a tympanic membrane of the ear of the subject.

22. The apparatus of claim 11, further comprising an ear hook coupled to the housing.

23. A method for measuring a physiological parameter of a subject, the method comprising:
(a) placing a health monitor inside an ear of the subject, the health monitor comprising an array comprising a plurality of piezoelectric micromachined ultrasonic transducers (pMUTs) and a plurality of optical units each comprising a light-emitting diode (LED) and an optical sensor;
(b) producing at least one of near-infrared light or mid-infrared light using the LED of at least one of the plurality of optical units;
(c) generating an image of blood vessels in the ear of the subject using at least one of the plurality of pMUTs or the plurality of optical units; and
(d) analyzing the image to determine a physiologic parameter of the subject.

24. The method of claim 23, further comprising:
modulating a parameter of the at least one of near-infrared light or the mid-infrared light,
producing the at least one of near infrared light or mid infrared light based on modulating the parameter and using the LED of at least one of the plurality of optical units,
generating a second image of blood vessels in the ear of the subject using the at least one of the plurality of pMUTs or the plurality of optical units, and
comparing the second image with the image to determine a physiologic parameter of the subject.

25. The method of claim 23, wherein the LED of at least one of the plurality of optical units is configured to provide spectroscopic imaging.

26. The method of claim 23, wherein the LED of at least one of the plurality of optical units is configured to perturb a tissue within the ear of the subject, and
wherein analyzing the image further comprises:
measuring vibrations through the tissue in response to perturbing the tissue within the ear of the subject.

27. The method of claim 23, further comprising:
repeating steps (b) and (c) a plurality of times to produce a plurality of images, and
wherein analyzing the image further comprises:
averaging the plurality of images to provide an averaged image, and
analyzing the averaged image.

28. The method of claim 23, wherein producing at least one of near-infrared light or mid-infrared light further comprises:
producing a plurality of pulses of the at least one of near infrared light or mid infrared light,
wherein each pulse has a duration between 5-300 nanoseconds and an energy between 10-500 microjoules.

29. The method of claim 23, wherein the health monitor further comprises one or more electrodes, and
wherein the method further comprises:
applying electrical stimulation configured to stimulate a nerve of the subject.

30. The method of claim 29, wherein analyzing the image to determine a physiologic parameter further comprises:
analyzing a change in blood flow in response to the electrical stimulation.

31. The method of claim 29, wherein analyzing the image to determine a physiologic parameter further comprises:
analyzing a change in a concentration of a blood biomarker in response to the electrical stimulation.

32. The method of claim 31, wherein the biomarker is at least one of glucose, a cytokine, oxygen, or white blood cell.

33. The method of claim 23, wherein analyzing the image to determine a physiologic parameter further comprises:
analyzing a change in blood flow.

34. The method of claim 23, wherein analyzing the image to determine a physiologic parameter further comprises:
analyzing a change in a concentration of a blood biomarker.

35. The method of claim 34, wherein the biomarker is at least one of glucose, a cytokine, oxygen, or white blood cell.
